(19) United States
(12) Patent Application Publication
(10) Pub. No.: US 2025/0268665 A1
(43) Pub. Date: Aug. 28, 2025

(54) ELONGATE INSTRUMENT WITH PROXIMAL POSE AND SHAPE SENSING

(71) Applicant: Auris Health, Inc., Santa Clara, CA (US)

(72) Inventors: Christopher Sramek, Half Moon Bay, CA (US); William Lanphier Chapin, Redwood City, CA (US); Namita Anil Kumar, Redwood City, CA (US)

(73) Assignee: Auris Health, Inc., Santa Clara, CA (US)

(21) Appl. No.: 19/060,586

(22) Filed: Feb. 21, 2025

Related U.S. Application Data
(60) Provisional application No. 63/557,442, filed on Feb. 23, 2024.

(51) Int. Cl. A61B 34/00; A61B 34/20; A61B 34/30; A61M 25/01

(52) U.S. Cl. CPC A61B 34/25; A61B 2034/2051; A61B 2034/2061; A61B 2034/303; A61M 25/0127

(57) ABSTRACT

This disclosure provides methods, devices, and systems for localizing medical instruments. The present implementations more specifically relate to techniques for localizing a distal tip of an elongate medical instrument based at least in part on first sensor data received from one or more first sensors disposed in a proximal hub of the instrument. For example, the first sensor data may indicate a position and/or orientation of the proximal hub, which can be used to determine a position and/or orientation of the distal tip based on a known length of the instrument. In some implementations, the controller may further determine a shape of the instrument based on second sensor data received from one or more second sensors disposed on a shaft and/or distal tip of the instrument. In such implementations, the position and/or orientation of the distal portion may be further determined based on the shape of the instrument.

### CROSS-REFERENCE TO RELATED APPLICATIONS

This application claims priority and benefit under 35 U.S.C. § 119(e) to U.S. Provisional Patent Application No. 63/557,442, filed Feb. 23, 2024, which is incorporated herein by reference in its entirety.

### TECHNICAL FIELD

This disclosure relates generally to medical robotics, and specifically to elongate instruments with proximal pose and shape sensing.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, in a PCNL procedure, a physician can insert a ureteroscope into the urinary tract through the urethra. A ureteroscope includes an endoscope at its distal end configured to enable visualization of the urinary tract. The physician navigates the ureteroscope through the bladder, up the ureter, and into the kidney where a kidney stone is located. Once at the site of a kidney stone (such as within a calyx of the kidney), the ureteroscope can be used to designate or “tag” a target location for a second medical instrument (such as a needle) to access the kidney percutaneously. The physician drives the needle into the patient, through the target location, and uses another medical instrument (which may be in conjunction with the needle) to extract the kidney stone from the patient via the percutaneous access point. The success or failure of a percutaneous medical procedure often depends on various factors, including the physician's skill, the patient's anatomy, as well as the precision and accuracy of any tools or equipment the physician uses to perform the procedure.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a system including an instrument, a sensor disposed on a proximal portion of the instrument, and a controller. The instrument has a distal portion configured to be inserted into an anatomy. The controller is configured to receive sensor data from the sensor indicating a position or orientation of the proximal portion of the instrument and determine a position or orientation of the distal portion of the instrument based at least in part on the received sensor data and a known length of the instrument.

Another innovative aspect of the subject matter of this disclosure can be implemented in a method for localizing medical instruments. The method includes steps of receiving sensor data from a sensor disposed on an instrument having a distal portion configured to be inserted into an anatomy, where the sensor data indicates a position or orientation of a proximal portion of the instrument; and determining a position or orientation of the distal portion of the instrument based at least in part on the received sensor data and a known length of the instrument.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a top view of the medical system of FIG. 1 configured to assist in inserting a scope into a patient.

FIG. 3 shows a top view of the medical system of FIG. 1 configured to navigate a scope within a patient.

FIG. 4 shows a top view of medical system of FIG. 1 configured to assist in inserting a needle into a patient.

FIG. 5 shows an example medical instrument, according to some implementations.

FIG. 6 shows another example medical instrument, according to some implementations.

FIG. 7 shows another example medical instrument, according to some implementations.

FIG. 8 shows a block diagram of an example controller for a medical system, according to some implementations.

FIG. 9 shows an illustrative flowchart depicting an example operation for localizing medical instruments, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope and/or a percutaneous access needle) to access a target site within an anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as a percutaneous access instrument that is exclusively controlled and operated by a physician). The systems and techniques described herein also may be applicable beyond the context of medical procedures (such as in simulated environments or laboratory settings, such as with models or simulators, among other examples).

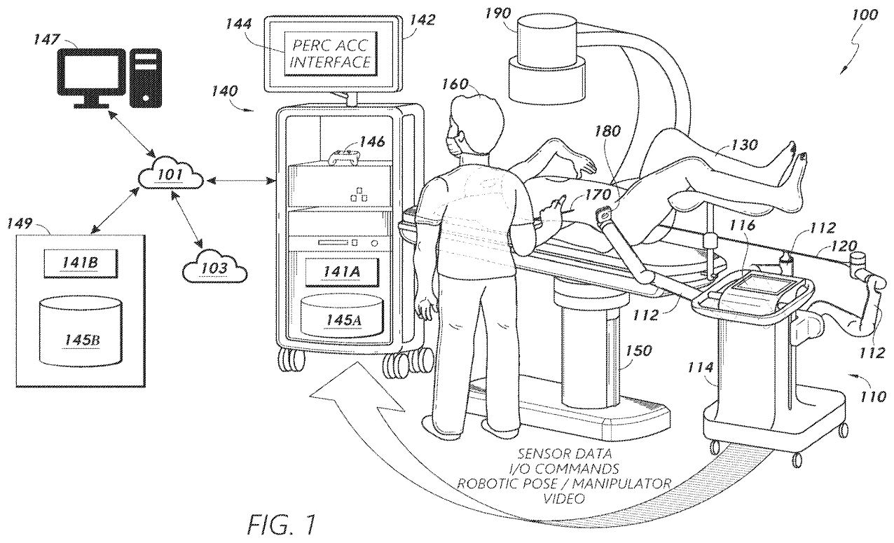

FIG. 1 shows an example medical system 100, according to some implementations. The medical system 100 includes a robotic system 110 configured to engage with or control a medical instrument to perform a procedure on a patient 130. The medical system 100 also includes a control system 140 configured to interface with the robotic system 110, provide information regarding the procedure, or perform a variety of other operations. For example, the control system 140 can include a display(s) 142 to present certain information to assist the physician 160. The display(s) 142 may be a monitor, screen, television, virtual reality hardware, augmented reality hardware, or three-dimensional imaging devices (such as hologram devices), among other examples, or combinations thereof. The medical system 100 can include a table 150 configured to hold the patient 130. The system 100 can further include an electromagnetic (EM) field generator 180, which can be held by one or more robotic arms 112 of the robotic system 110 or can be a stand-alone device. In examples, the medical system 100 can also include an imaging device 190 which can be integrated into a C-arm or configured to provide imaging during a procedure, such as for a fluoroscopy-type procedure. Although shown in FIG. 1, in some implementations the imaging device 190 is eliminated.

In some implementations, the medical system 100 can be used to perform a percutaneous procedure. For example, if the patient 130 has a kidney stone that is too large to be removed through a urinary tract, the physician 160 can perform a procedure to remove the kidney stone through a percutaneous access point on the patient 130. To illustrate, the physician 160 can interact with the control system 140 to control the robotic system 110 to advance and navigate a first medical instrument (such as a scope) from the urethra, through the bladder, up the ureter, and into the kidney where the stone is located. The control system 140 can provide information via the display(s) 142 regarding the first medical instrument to assist the physician 160 in navigating the first medical instrument, such as real-time images captured therewith.

Once at the site of the kidney stone (such as within a calyx of the kidney), the first medical instrument can be used to designate or tag a target location for a second medical instrument (such as a needle) to access the kidney percutaneously (such as a desired point to access the kidney). To minimize damage to the kidney or the surrounding anatomy, the physician 160 can designate a particular papilla as the target location for entering into the kidney with the second medical instrument. However, other target locations can be designated or determined. To assist the physician in driving the second medical instrument into the patient 130 through the particular papilla, the control system 140 can provide a percutaneous access interface 144, which can include a visualization to indicate an alignment of an orientation of the second medical instrument relative to a target trajectory (such as a desired access path from the patient's skin to the target location), a visualization to indicate a progress of inserting the second medical instrument into the kidney towards the target location, guidance on the percutaneous procedure, or other information. Once the second medical instrument has reached the target location (as determined, such as by sensors attached to the needle 170, the scope 120, or other any sensor or imaging modality), the physician 160 can use the second medical instrument or another medical instrument to extract the kidney stone from the patient 130, such as through the percutaneous access point.

Although the above percutaneous procedure or other procedures are discussed in the context of using the first medical instrument, in some implementations a percutaneous procedure can be performed without the assistance of the first medical instrument. Further, the medical system 100 can be used to perform a variety of other procedures. Moreover, although many implementations describe the physician 160 using the second medical instrument, the second medical instrument can alternatively be used by a component of the medical system 100. For example, the second medical instrument can be held or manipulated by the robotic system 110 (such as the one or more robotic arms 112) and the techniques discussed herein can be implemented to control the robotic system 110 to insert the second medical instrument with the appropriate pose (or aspect of a pose, such as orientation or position) to reach a target location.

In the example of FIG. 1, the first medical instrument is implemented as a scope 120 and the second medical instrument is implemented as a needle 170. Thus, for ease of discussion, the first medical instrument is referred to as “the scope 120” or “the lumen-based medical instrument,” and the second medical instrument is referred to as “the needle 170” or “the percutaneous medical instrument.” However, the first medical instrument and the second medical instrument can each be implemented as a suitable type of medical instrument including, for example, a scope (sometimes referred to as an “endoscope”), a needle, a catheter, a guidewire, a lithotripter, a basket retrieval device, forceps, a vacuum, a needle, a scalpel, an imaging probe, jaws, scissors, graspers, needle holder, micro dissector, staple applier, tacker, suction or irrigation tool, or clip applier, among other examples. In some implementations, a medical instrument is a steerable device, while some other implementations a medical instrument is a non-steerable device. In some implementations, a surgical tool refers to a device that is configured to puncture or to be inserted through the human anatomy, such as a needle, a scalpel, or a guidewire, among other examples. However, a surgical tool can refer to other types of medical instruments.

In some implementations, a medical instrument, such as the scope 120 or the needle 170, includes a sensor that is configured to generate sensor data, which can be sent to another device. In examples, sensor data can indicate a location or orientation of the medical instrument or can be used to determine a location or orientation of the medical instrument. For instance, a sensor can include an electromagnetic (EM) sensor with a coil of conductive material. Here, an EM field generator, such as the EM field generator 180, can provide an EM field that is detected by the EM sensor on the medical instrument. The magnetic field can induce small currents in coils of the EM sensor, which can be analyzed to determine a distance or angle or orientation between the EM sensor and the EM field generator. Further, a medical instrument can include other types of sensors configured to generate sensor data, such as one or more of any of: a camera, a range sensor, a radar device, a shape sensing fiber, an accelerometer, a gyroscope, a satellite-based positioning sensor (such as a global positioning system (GPS)), or a radio-frequency transceiver, among other examples. In some implementations, a sensor is positioned on a distal end of a medical instrument, while in some other implementations a sensor is positioned at another location on the medical instrument. In some implementations, a sensor on a medical instrument can provide sensor data to the control system 140 and the control system 140 can perform one or more localization techniques to determine or track a position or an orientation of a medical instrument.

In some implementations, the medical system 100 may record or otherwise track the runtime data that is generated during a medical procedure. For example, the medical system 100 may track or otherwise record the sensor readings (such as sensor data) from the instruments (such as the scope 120 and the needle 170) in case data store 145A (such as a computer storage system, such as computer readable memory, database, or filesystem, among other examples). In addition to sensor data, the medical system 100 can store other types of case logs in the case data store 145A. For example, in the context of FIG. 1, the case logs can include time series data of the video images captured by the scope 120, status of the robotic system 110, commanded data from an I/O device(s) (such as I/O device(s) 146), audio data (such as may be captured by audio capturing devices embedded in the medical system 100, such as microphones on the medical instruments, robotic arms, or elsewhere in the medical system), external (relative to the patient) imaging device (such as RGB cameras, LIDAR imaging sensors, or fluoroscope imaging sensors), among other examples.

As shown in FIG. 1, the control system 140 includes an analytics engine 141A which may operate on the case logs stored in the case data store 145A to label the case logs according to a procedure phase for a given time. In some implementations, the analytics engine 141A may employ machine learning techniques to segment the medical procedure according to the different phases. In some implementations, once the medical procedure has been segmented, the analytics engine 141A may generate metrics for the medical procedure phases, the medical procedure generally, or provide insights and recommendations to the users of the medical system 100 (such as physicians, staff, or training personnel).

FIG. 1 further shows that in some implementations the control system 140 may include a network connection (such as via network 101) to a cloud-based data analytics platform 149. The cloud-based data analytics platform 149 may be a computer system that provides third party computers 147 postoperative analytic capabilities on a given medical procedure or analytics across multiple medical procedures. As shown in FIG. 1, the cloud-based data analytics platform 149 may further connect to additional medical systems 103, which each in turn may transmit case logs to the cloud-based data analytics platform 149. Because the cloud-based data analytics platform receives case logs from multiple medical systems, the cloud-based data analytics platform 149 may have access to a comparatively larger pool of data than a single medical system would have access to and may in turn aggregate the case logs across multiple medical systems to derive medical procedure insights. Medical procedure insights may include guidance on factors that result in an increased likelihood for success in a medical procedure based on the metrics derived from segmenting the case logs across medical systems and across medical procedures.

As shown in FIG. 1, the cloud-based data analytics platform 149 may include an analytics engine 141B and cloud-based data store 145B. The cloud-based data store 145B may be a computer storage device that stores the system data records from the medical system 100 and the additional medical systems 103. The analytics engine 141B may include features and capabilities similar to the analytics engine 141A. However, in some implementations, the analytics engine 141A may further operate to analyze case logs across multiple medical systems (such as medical system 100 and medical systems 103) to generate metrics or insights. This may provide comparatively robust insights because the data used to generate such metrics or insights is using a broader range of information. Additionally, or alternatively, the cloud-based analytics engine 141B may uses machine learning techniques that are suitable for post-operative classification, whereas the local analytics engine 141B may use machine learning techniques that are suitable for real-time or near-real time classification.

The term “scope” or “endoscope” is used herein according to its broad and ordinary meanings and can refer to any type of elongate medical instrument having image generating, viewing, or capturing functionality and configured to be introduced into any type of organ, cavity, lumen, chamber, or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), or a borescope, among other examples.

A scope can comprise a tubular or flexible medical instrument that is configured to be inserted into the anatomy of a patient to capture images of the anatomy. In some implementations, a scope can accommodate wires or optical fibers to transfer signals to or from an optical assembly and a distal end of the scope, which can include an imaging device, such as an optical camera. The camera or imaging device can be used to capture images of an internal anatomical space, such as a target calyx or papilla of a kidney. A scope can further be configured to accommodate optical fibers to carry light from proximately-located light sources, such as light-emitting diodes, to the distal end of the scope. The distal end of the scope can include ports for light sources to illuminate an anatomical space when using the camera or imaging device. In some implementations, the scope is configured to be controlled by a robotic system, such as the robotic system 110. The imaging device can comprise an optical fiber, fiber array, or lens. The optical components can move along with the tip of the scope such that movement of the tip of the scope results in changes to the images captured by the imaging device.

A scope can be articulable, such as with respect to at least a distal portion of the scope, so that the scope can be steered within the human anatomy. In some implementations, a scope is configured to be articulated with, for example, five or six degrees of freedom, including X, Y, Z coordinate movement, as well as pitch, yaw, and roll. A position sensor(s) of the scope can likewise have similar degrees of freedom with respect to the position information they produce or provide. A scope can include telescoping parts, such as an inner leader portion and an outer sheath portion, which can be manipulated to telescopically extend the scope. A scope, in some instances, can comprise a rigid or flexible tube, and can be dimensioned to be passed within an outer sheath, catheter, introducer, or other lumen-type device, or can be used without such devices. In some implementations, a scope includes a working channel for deploying medical instruments (such as lithotripters, basketing devices, or forceps), irrigation, or aspiration to an operative region at a distal end of the scope.

The robotic system 110 can be configured to at least partly facilitate execution of a medical procedure. The robotic system 110 can be arranged in a variety of ways depending on the particular procedure. The robotic system 110 can include the one or more robotic arms 112 configured to engage with or control the scope 120 to perform a procedure. As shown, each robotic arm 112 can include multiple arm segments coupled to joints, which can provide multiple degrees of movement. In the example of FIG. 1, the robotic system 110 is positioned proximate to the patient's 130 legs and the robotic arms 112 are actuated to engage with and position the scope 120 for access into an access point, such as the urethra of the patient 130. When the robotic system 110 is properly positioned, the scope 120 can be inserted into the patient 130 robotically using the robotic arms 112, manually by the physician 160, or a combination thereof. The robotic arms 112 can also be connected to the EM field generator 180, which can be positioned near a treatment site, such as within proximity to the kidneys of the patient 130.

The robotic system 110 can also include a support structure 114 coupled to the one or more robotic arms 112. The support structure 114 can include control electronics or circuitry, one or more power sources, one or more pneumatics, one or more optical sources, one or more actuators (such as motors to move the one or more robotic arms 112), memory or data storage, or one or more communication interfaces. In some implementations, the support structure 114 includes an input/output (I/O) device(s) 116 configured to receive input, such as user input to control the robotic system 110, or provide output, such as a graphical user interface (GUI), information regarding the robotic system 110, or information regarding a procedure, among other examples. The I/O device(s) 116 can include a display, a touchscreen, a touchpad, a projector, a mouse, a keyboard, a microphone, or a speaker. In some implementations, the robotic system 110 is movable (such as the support structure 114 includes wheels) so that the robotic system 110 can be positioned in a location that is appropriate or desired for a procedure. In some other implementations, the robotic system 110 is a stationary system. Further, in some implementations, the robotic system 110 is integrated into the table 150.

The robotic system 110 can be coupled to any component of the medical system 100, such as the control system 140, the table 150, the EM field generator 180, the scope 120, or the needle 170. In some implementations, the robotic system is communicatively coupled to the control system 140. In one example, the robotic system 110 can be configured to receive a control signal from the control system 140 to perform an operation, such as to position a robotic arm 112 in a particular manner, or manipulate the scope 120, among other examples. In response, the robotic system 110 can control a component of the robotic system 110 to perform the operation. In another example, the robotic system 110 is configured to receive an image from the scope 120 depicting internal anatomy of the patient 130 or send the image to the control system 140, which can then be displayed on the display(s) 142. Furthermore, in some implementations, the robotic system 110 is coupled to a component of the medical system 100, such as the control system 140, in such a manner as to allow for fluids, optics, or power, among other examples, to be received therefrom.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110 or the scope 120, receive an image(s) captured by the scope 120), provide fluids to the robotic system 110 via one or more fluid channels, provide power to the robotic system 110 via one or more electrical connections, provide optics to the robotic system 110 via one or more optical fibers or other components, among other examples. Further, in some implementations, the control system 140 can communicate with the needle 170 or the scope 120 to receive sensor data from the needle 170 or the scope 120 (via the robotic system 110 or directly from the needle 170 or the scope 120). Moreover, in some implementations, the control system 140 can communicate with the table 150 to position the table 150 in a particular orientation or otherwise control the table 150. Further, in some implementations, the control system 140 can communicate with the EM field generator 180 to control generation of an EM field around the patient 130.

The control system 140 includes various I/O devices configured to assist the physician 160 or others in performing a medical procedure. In this example, the control system 140 includes an I/O device(s) 146 that is employed by the physician 160 or other user to control the scope 120, such as to navigate the scope 120 within the patient 130. For example, the physician 160 can provide input via the I/O device(s) 146 and, in response, the control system 140 can send control signals to the robotic system 110 to manipulate the scope 120. Although the I/O device(s) 146 is illustrated as a controller in the example of FIG. 1, the I/O device(s) 146 can be implemented as a variety of types of I/O devices, such as a touchscreen, a touch pad, a mouse, a keyboard, a surgeon or physician console, virtual reality hardware, augmented hardware, microphone, speakers, or haptic devices, among other examples.

As also shown in FIG. 1, the control system 140 can include the display(s) 142 to provide various information regarding a procedure. The display(s) 142 can present the percutaneous access interface 144 to assist the physician 160 in the percutaneous access procedure (such as manipulating the needle 170 towards a target site). The display(s) 142 can also provide (such as via the percutaneous access interface 144 or another interface) information regarding the scope 120. For example, the control system 140 can receive real-time images that are captured by the scope 120 and display the real-time images via the display(s) 142. Additionally, or alternatively, the control system 140 can receive signals (such as analog, digital, electrical, acoustic or sonic, pneumatic, tactile, hydraulic) from a medical monitor or a sensor associated with the patient 130, and the display(s) 142 can present information regarding the health or environment of the patient 130. Such information can include information that is displayed via a medical monitor including, for example, a heart rate (such as ECG or HRV), blood pressure or rate, muscle bio-signals (such as EMG), body temperature, blood oxygen saturation (such as SpO2), CO2, brainwaves (such as EEG), environmental or local or core body temperature, among other examples.

To facilitate the functionality of the control system 140, the control system 140 can include various components (sometimes referred to as “subsystems”). For example, the control system 140 can include control electronics or circuitry, as well as one or more power sources, pneumatics, optical sources, actuators, memory or data storage devices, or communication interfaces. In some implementations, the control system 140 includes control circuitry comprising a computer-based control system that is configured to store executable instructions, that when executed, cause various operations to be implemented. In some implementations, the control system 140 is movable, such as that shown in FIG. 1, while in some other implementations, the control system 140 is a stationary system. Although various functionality and components are discussed as being implemented by the control system 140, any of this functionality or components can be integrated into or performed by other systems or devices, such as the robotic system 110, the table 150, or the EM field generator 180 (or even the scope 120 or the needle 170).

The imaging device 190 can be configured to capture or generate one or more images of the patient 130 during a procedure, such as one or more x-ray or CT images. In examples, images from the imaging device 190 can be provided in real-time to view anatomy or medical instruments, such as the scope 120 or the needle 170, within the patient 130 to assist the physician 160 in performing a procedure. The imaging device 190 can be used to perform a fluoroscopy (such as with a contrast dye within the patient 130) or another type of imaging technique. Although shown in FIG. 1, in many implementations the imaging device 190 is not implemented for performing a procedure or the imaging device 190 (including the C-arm) is eliminated.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, or the Internet. Further, in some implementations, the components of the medical system 100 are connected for data communication, fluid or gas exchange, or power exchange, among other examples, via one or more support cables, or tubes, among other examples.

The medical system 100 can provide a variety of benefits, such as providing guidance to assist a physician in performing a procedure (such as instrument tracking or instrument alignment information), enabling a physician to perform a procedure from an ergonomic position without the need for awkward arm motions or positions, enabling a single physician to perform a procedure with one or more medical instruments, avoiding radiation exposure (such as associated with fluoroscopy techniques), enabling a procedure to be performed in a single-operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding or damage to anatomy (such as critical organs or blood vessels). Further, the medical system 100 can provide non-radiation-based navigational or localization techniques to reduce physician and patient exposure to radiation or reduce the amount of equipment in the operating room. Moreover, the medical system 100 can provide functionality that is distributed between at least the control system 140 and the robotic system 110, which can be independently movable. Such distribution of functionality or mobility can enable the control system 140 or the robotic system 110 to be placed at locations that are optimal for a particular medical procedure, which can maximize working area around the patient or provide an optimized location for a physician to perform a procedure.

Although various techniques and systems are discussed as being implemented as robotically-assisted procedures (such as procedures that at least partly use the medical system 100), the techniques and systems can be implemented in other procedures, such as in fully-robotic medical procedures, or human-only procedures (such as free of robotic systems), among other examples. For example, the medical system 100 can be used to perform a procedure without a physician holding or manipulating a medical instrument (such as a fully-robotic procedure). That is, medical instruments that are used during a procedure, such as the scope 120 and the needle 170, can each be held or controlled by components of the medical system 100, such as the robotic arm(s) 112 of the robotic system 110.

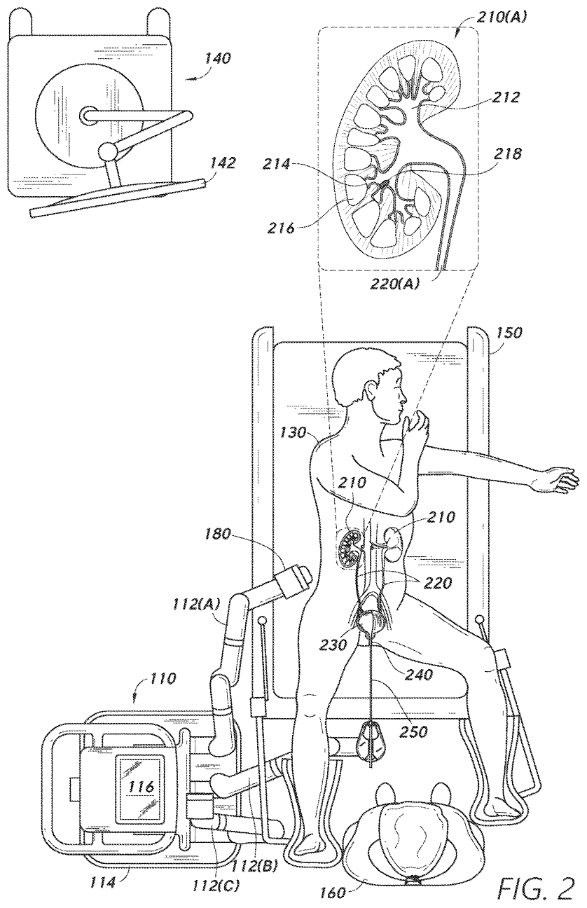

FIGS. 2-4 illustrate a top view of the medical system 100 of FIG. 1 arranged to perform a percutaneous procedure in accordance with one or more implementations. In these examples, the medical system 100 is arranged in an operating room to remove a kidney stone from the patient 130 with the assistance of the scope 120 and the needle 170. In some implementations of such a procedure, the patient 130 is positioned in a modified supine position with the patient 130 slightly tilted to the side to access the flank of the patient 130, such as that illustrated in FIG. 1. However, the patient 130 can be positioned in other manners, such as a supine position, or a prone position, among other examples. For ease of illustration in viewing the anatomy of the patient 130, FIG. 2-4 illustrate the patient 130 in a supine position with the legs spread apart. Also, for ease of illustration, the imaging device 190 (including the C-arm) has been removed.

Although FIGS. 2-4 illustrate use of the medical system 100 to perform a percutaneous procedure to remove a kidney stone from the patient 130, the medical system 100 can be used to remove a kidney stone in other manners or to perform other procedures. Further, the patient 130 can be arranged in other positions as desired for a procedure. Various acts are described in FIGS. 2-4 and throughout this disclosure as being performed by the physician 160. It should be understood that these acts can be performed directly by the physician 160, a user under direction of the physician, another user (such as a technician), a combination thereof, or any other user.

FIGS. 2-4 show various features of the anatomy of the patient 130. For example, the patient 130 includes kidneys 210 fluidly connected to a bladder 230 via ureters 220, and a urethra 240 fluidly connected to the bladder 230. As shown in the enlarged depiction of the kidney 210(A), the kidney 210(A) includes calyces (including calyx 212), renal papillae (including the renal papilla 214, also referred to as “the papilla 214”), and renal pyramids (including the renal pyramid 216). In these examples, a kidney stone 218 is located in proximity to the papilla 214. However, the kidney stone 218 can be located at other locations within the kidney 210(A) or elsewhere.

As shown in FIG. 2, to remove the kidney stone 218 in the example percutaneous procedure, the physician 160 can position the robotic system 110 at the side or foot of the table 150 to initiate delivery of the scope 120 (not illustrated in FIG. 2) into the patient 130. In particular, the robotic system 110 can be positioned at the side of the table 150 within proximity to the feet of the patient 130 and aligned for direct linear access to the urethra 240 of the patient 130. In examples, the hip of the patient 130 is used as a reference point to position the robotic system 110. Once positioned, one or more of the robotic arms 112, such as the robotic arms 112(B) and 112(C), can stretch outwards to reach in between the legs of the patient 130. For example, the robotic arm 112(B) can be controlled to extend and provide linear access to the urethra 240, as shown in FIG. 2. In this example, the physician 160 inserts a medical instrument 250 at least partially into the urethra 240 along this direct linear access path (sometimes referred to as “a virtual rail”). The medical instrument 250 can include a lumen-type device configured to receive the scope 120, thereby assisting in inserting the scope 120 into the anatomy of the patient 130. By aligning the robotic arm 112(B) to the urethra 240 of the patient 130 or using the medical instrument 250, friction or forces on the sensitive anatomy in the area can be reduced. Although the medical instrument 250 is illustrated in FIG. 2, in some implementations, the medical instrument 250 is not used (such as the scope 120 can be inserted directly into the urethra 240).

The physician 160 can also position the robotic arm 112(A) near a treatment site for the procedure. For example, the robotic arm 112(A) can be positioned within proximity to the incision site or the kidneys 210 of the patient 130. The robotic arm 112(A) can be connected to the EM field generator 180 to assist in tracking a location of the scope 120 or the needle 170 during the procedure. Although the robotic arm 112(A) is positioned relatively close to the patient 130, in some implementations the robotic arm 112(A) is positioned elsewhere or the EM field generator 180 is integrated into the table 150 (which can allow the robotic arm 112(A) to be in a docked position). In this example, at this point in the procedure, the robotic arm 112(C) remains in a docked position, as shown in FIG. 2. However, the robotic arm 112(C) can be used in some implementations to perform any of the functions of the robotic arms 112(A) or 112(C).

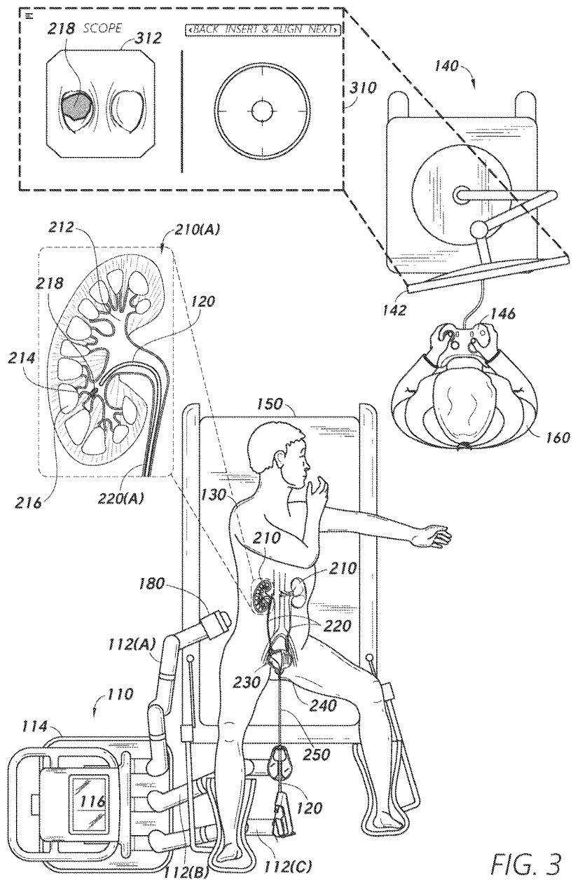

Once the robotic system 110 is properly positioned or the medical instrument 250 is inserted at least partially into the urethra 240, the scope 120 can be inserted into the patient 130 robotically, manually, or a combination thereof, as shown in FIG. 3. For example, the physician 160 can connect the scope 120 to the robotic arm 112(C) or position the scope 120 at least partially within the medical instrument 250 or the patient 130. The scope 120 can be connected to the robotic arm 112(C) at any time, such as before the procedure or during the procedure (such as after positioning the robotic system 110). The physician 160 can then interact with the control system 140, such as the I/O device(s) 146, to navigate the scope 120 within the patient 130. For example, the physician 160 can provide input via the I/O device(s) 146 to control the robotic arm 112(C) to navigate the scope 120 through the urethra 240, the bladder 230, the ureter 220(A), and up to the kidney 210(A).

As shown, the control system 140 can present an instrument-alignment interface 310, such as the instrument-alignment interface 310 of FIG. 3, via the display(s) 142 to view a real-time image 312 captured by the scope 120 to assist the physician 160 in controlling the scope 120. The physician 160 can navigate the scope 120 to locate the kidney stone 218, as depicted in the image 312. In some implementation, the control system 140 can use localization techniques to determine a position or an orientation of the scope 120, which can be viewed by the physician 160 through the display(s) 142 (not illustrated on the display(s) 142 in FIG. 3) to also assist in controlling the scope 120. Further, in some implementations, other types of information can be presented through the display(s) 142 to assist the physician 160 in controlling the scope 120, such as x-ray images of the internal anatomy of the patient 130.

Upon locating the kidney stone 218, the physician 160 can identify a location for the needle 170 to enter the kidney 210(A) for eventual extraction of the kidney stone 218. For example, to minimize bleeding or avoid hitting a blood vessel or other undesirable anatomy of the kidney 210(A) or anatomy surrounding the kidney 210(A), the physician 160 can seek to align the needle 170 with an axis of a calyx (such as can seek to reach the calyx head-on through the center of the calyx). To do so, the physician 160 can identify a papilla as a target location. In this example, the physician 160 uses the scope 120 to locate the papilla 214 that is near the kidney stone 218 and designate the papilla 214 as the target location. In some implementations of designating the papilla 214 as the target location, the physician can cause the medical system to tag the papilla. In tagging the papilla, the physician 160 can navigate the scope 120 to contact the papilla 214 and provide a UI input to the system to indicate the tagging, the control system 140 can use localization techniques to determine a location of the scope 120 (such as a location of the end of the scope 120), and the control system 140 can associate the location of the scope 120 with the target location. Additionally, or alternatively, the physician 160 can navigate the scope 120 to be within a particular distance to the papilla 214 (such as park in front of the papilla 214) and provide input indicating that the target location is within a field-of-view of the scope 120. The control system 140 can perform image analysis or other localization techniques to determine a location of the target location. In some other implementations, the scope 120 can deliver a fiduciary to mark the papilla 214 as the target location.

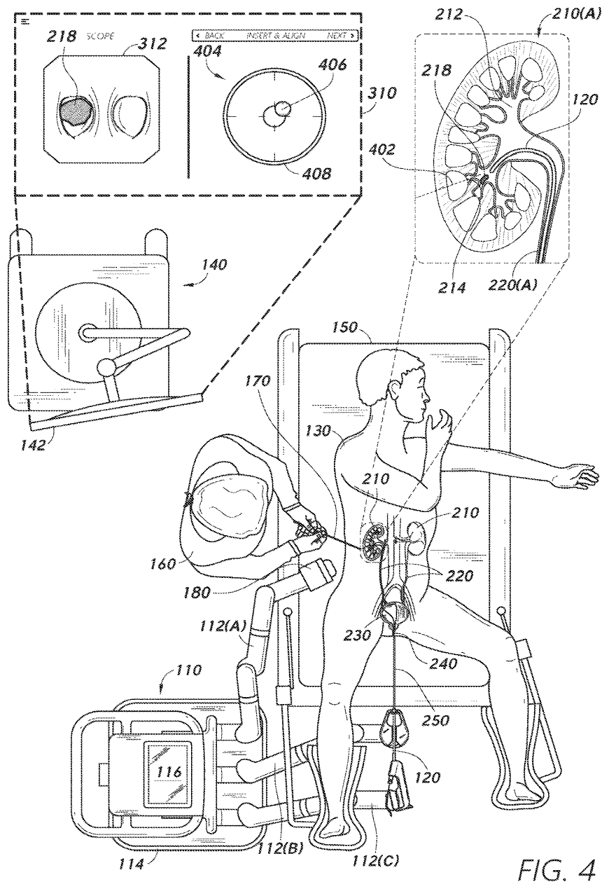

As shown in FIG. 4, the physician 160 can proceed with the procedure by positioning the needle 170 for insertion into the target location. In some implementations, the physician 160 can use his or her best judgment to place the needle 170 on the patient 130 at an incision site, such as based on knowledge regarding the anatomy of the patient 130, experience from previously performing the procedure, an analysis of CT or x-ray images or other pre-operative information of the patient 130, among other examples. Further, in some implementations, the control system 140 can provide information regarding a location to place the needle 170 on the patient 130. The physician 160 can attempt to avoid critical anatomy of the patient 130, such as the colon, paraspinal muscles, ribs, intercostal nerves, lungs, or pleura. In some examples, the control system 140 can use CT, x-ray, or ultrasound images to provide information regarding a location to place the needle 170 on the patient 130.

The control system 140 can determine a target trajectory 402 for inserting the needle 170 to assist the physician 160 in reaching the target location (such as the papilla 214). The target trajectory 402 can represent a desired path for accessing the target location. The target trajectory 402 can be determined based on a position of a medical instrument (such as the needle 170 or the scope 120), a target location within the human anatomy, a position or orientation of a patient, or the anatomy of the patient (such as the location of organs within the patient relative to the target location), among other examples. In this example, the target trajectory 402 includes a straight line that passes through the papilla 214 and the needle 170 (such as extends from a tip of the needle 170 through the papilla 214, such as a point on an axis of the papilla 214). However, the target trajectory 402 can take other forms, such as a curved line, or can be defined in other manners. In some examples, the needle 170 is implemented a flexible bevel-tip needle that is configured to curve as the needle 170 is inserted in a straight manner. Such needle can be used to steer around particular anatomy, such as the ribs or other anatomy. Here, the control system 140 can provide information to guide a user, such as to compensate for deviation in the needle trajectory or to maintain the user on the target trajectory.

Although the example of FIG. 4 illustrates the target trajectory 402 extending coaxially through the papilla 214, the target trajectory 402 can have another position, angle, or form. For example, a target trajectory can be implemented with a lower pole access point, such as through a papilla located below the kidney stone 218 shown in FIG. 4, with a non-coaxial angle through the papilla, which can be used to avoid the hip.

The control system 140 can use the target trajectory 402 to provide an alignment-progress visualization 404 via the instrument-alignment interface 310. For example, the alignment-progress visualization 404 can include an instrument alignment element 406 indicative of an orientation of the needle 170 relative to the target trajectory 402. The physician 160 can view the alignment-progress visualization 404 and orient the needle 170 to the appropriate orientation (such as the target trajectory 402). When aligned, the physician 160 can insert the needle 170 into the patient 130 to reach the target location. The alignment-progress visualization 404 can provide a progress visualization 408 (also referred to as “the progress bar 408”) indicative of a proximity of the needle 170 to the target location. As such, the instrument-alignment interface 310 can assist the physician 160 in aligning or inserting the needle 170 to reach the target location.

Once the target location has been reached with the needle 170, the physician 160 can insert another medical instrument, such as a power catheter, vacuum, or nephroscope into the path created by the needle 170 or over the needle 170. The physician 160 can use the other medical instrument or the scope 120 to fragment and remove pieces of the kidney stone 218 from the kidney 210(A).

In some implementations, a position of a medical instrument can be represented with a point, point set, or an orientation of the medical instrument can be represented as an angle or offset relative to an axis or plane. For example, a position of a medical instrument can be represented with a coordinate(s) of a point or point set within a coordinate system (such as one or more X, Y, Z coordinates) or an orientation of the medical instrument can be represented with an angle relative to an axis or plane for the coordinate system (such as angle with respect to the X-axis or plane, Y-axis or plane, or Z-axis or plane). Here, a change in orientation of the medical instrument can correspond to a change in an angle of the medical instrument relative to the axis or plane. Further, in some implementations, an orientation of a medical instrument is represented with yaw, pitch, or roll information. In some other implementations, an orientation of a medical instrument is represented in quaternion representation. Quaternion representation may avoid singularities present in a representation based on yaw, pitch, and roll.

In some implementations, a trajectory may refer to a pose. For example, a trajectory of a medical instrument can refer to a pose of the medical instrument, including or indicating both a position and orientation of the medical instrument. Similarly, a target trajectory can refer to a target pose, including or indicating both a position and orientation of a desired path. However, in some other implementations, a trajectory refers to either an orientation or a position.

Although particular robotic arms of the robotic system 110 are illustrated (or described herein) as performing particular functions in the context of FIGS. 2-4, any of the robotic arms 112 can be used to perform the functions. Further, any additional robotic arms or systems can be used to perform the procedure. Moreover, the robotic system 110 can be used to perform other parts of the procedure. For example, the robotic system 110 can be controlled to align or insert the needle into the patient 130. To illustrate, one of the robotic arms 112 can engage with or control the needle 170 to position the needle 170 at the appropriate location, align the needle 170 with the target trajectory, or insert the needle 170 to the target location. The control system 140 can use localization techniques to perform such processing. As such, in some implementations, a percutaneous procedure can be performed entirely or partially with the medical system 100 (such as with or without the assistance of the physician 160).

As described with reference to FIGS. 1-4, many medical instruments used in robotically assisted medical procedures include positioning sensors (such as EM sensors) that can be used for positioning or navigating the instruments inside a patient's body. For example, in existing implementations of a percutaneous access needle (such as the needle 170), an EM sensor is disposed in the needle tip which allows the medical system to track or monitor the movement and location of the tip in the presence of electromagnetic fields. However, in such implementations, the design of the sensors (such as size, type, sensitivity, or features) is limited by the size of the trocar (tip) or cannula (shaft) of the needle. For example, the small form factor generally limits tip-based sensors to long, small-diameter coils which can have low sensitivity. As a result, existing tip-based sensors are susceptible to distortion from conductive surfaces in the tracking system's working volume as well as electrical noise pick-up.

Further, while an EM sensor in the tip of the needle may be suitable for guiding the tip to rendezvous with a scope, it cannot provide any information about the shape or deflection of the needle. Aspects of the present disclosure recognize that some medical instruments, such as percutaneous access needles, have elongate shafts that can bend or deflect as they are pushed into a patient's body. Understanding how the shaft interacts with the patient's anatomy and deflects relative to the initial insertion site could improve accuracy in registration with respect to CT imaging to assist the physician in avoiding critical anatomy and improve their understanding of the spatial relationships between the insertion site and the tip of the shaft. Aspects of the present disclosure recognize that the design of such medical instruments can be improved by implementing positioning sensors or other circuitry into the “hub” of the instrument (such as the proximal portion, opposite the tip, where the physician or robot holds or otherwise interfaces with the instrument). Although specific reference is made herein to percutaneous access needles, aspects of the present disclosure can be applicable to any medical instrument having an elongate shaft of a predetermined length.

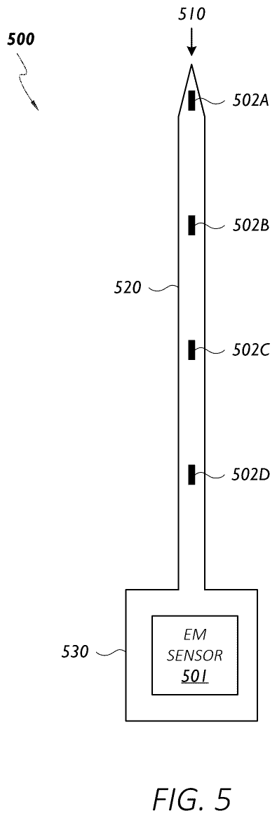

FIG. 5 shows an example medical instrument 500, according to some implementations. In the example of FIG. 5, the medical instrument 500 is depicted as a percutaneous access needle. In some implementations, the medical instrument 500 may be one example of the needle 170 of FIGS. 1-4.

The medical instrument 500 includes a trocar 510 (also referred to as the “tip” or “distal portion”), a cannula 520 (also referred to as the “shaft”), and a hub 530 which forms the base of the needle (also referred to as the “proximal portion”). In some implementations, the hub 530 may be a detachable and reusable component of the medical instrument 500, while the remainder of the medical instrument 500 (including the needle shaft 520 and tip 510) may be disposable. In some other implementations, the hub 530 may be permanently attached to the shaft 520 so that the medical instrument 500, as a whole, is disposable.

The hub 530 includes an EM sensor 501 that can be used for determining a position of the medical instrument 500 in the presence of magnetic fields (such as described with reference to FIGS. 1-4). In some implementations, the EM sensor 501 may produce sensor data indicating a position of the hub 530 with 6 degrees of freedom (such as by a position vector and quaternion). For example, the EM sensor 501 may include multiple inductive coils each positioned parallel to a different plane or axis of rotation. Alternatively, the EM sensor 501 can implement magnetic sensing technologies, such as magnetoresistance, rather than inductive coils, that can be used in combination with non-EM-based tracking technologies (for example, optical tracking). Because the medical instrument 500 has a known and/or fixed length, the position of the tip of the trocar 510 can be determined based, at least in part, on the position of the hub 530 and the direction that the hub 530 is facing (also referred to as the “orientation” of the hub 530). However, as described above, the cannula 520 may bend or deflect as the medical instrument 500 is driven to a target location. Thus, the shape of the cannula 520 affects the position of the trocar 510 relative to the position of the hub 530.

In some aspects, the medical instrument 500 may include one or more additional sensors 502A-502D disposed along the cannula 520 and/or the trocar 510. In the example of FIG. 5, the medical instrument 500 is shown to include 4 additional sensors 502A-502D. However, in actual implementations, the medical instrument 500 may include fewer or more additional sensors than those depicted in FIG. 5. In some implementations, the additional sensors 502A-502D may be disposed on an inner wall or surface of the medical instrument 500. In some other implementations, the additional sensors 502A-502D may be disposed on an outer surface of the medical instrument 500. The additional sensors 502A-502D may provide one or more additional sensing modalities. In some implementations, the additional sensors 502A-502D may produce sensor data that can be used to determine a shape or bend of the cannula 520. Such shape information can be combined with the position information produced by the EM sensor 501 to determine the position of the tip of the medical instrument 500 (or trocar 510) based on various mathematical models (such as Hermite spline or Bezier curve).

In some implementations, the additional sensors 502A-502D may include electromechanical strain gauges. For example, each strain gauge may be a flexible printed circuit (FPC) having fixed serpentine traces of equal separation. Changes in the strain of the traces create changes in resistance, which can be sensed via a multi-channel Wheatstone bridge proximal circuit configuration (not shown for simplicity). In some implementations, the FPC may be rolled inside the cannula 520, with sensor orientation perpendicular to the shaft direction, and positions around the needle circumference varying along the length of the cannula 520 (such as in trios located at approximately the same position along the length of the shaft). In some other implementations, the strain gauges may be directly deposited on the outer surface of the medical instrument 500 (similarly at various positions around the circumference along the length of the shaft). Such strain measurements, combined with the known locations of the additional sensors 502A-502D along the length of the medical instrument 500, enable the medical system to estimate the magnitude and direction of deflection of the instrument 500 based on a semi-rigid mechanical model (such as in accordance with 3D beam theory).

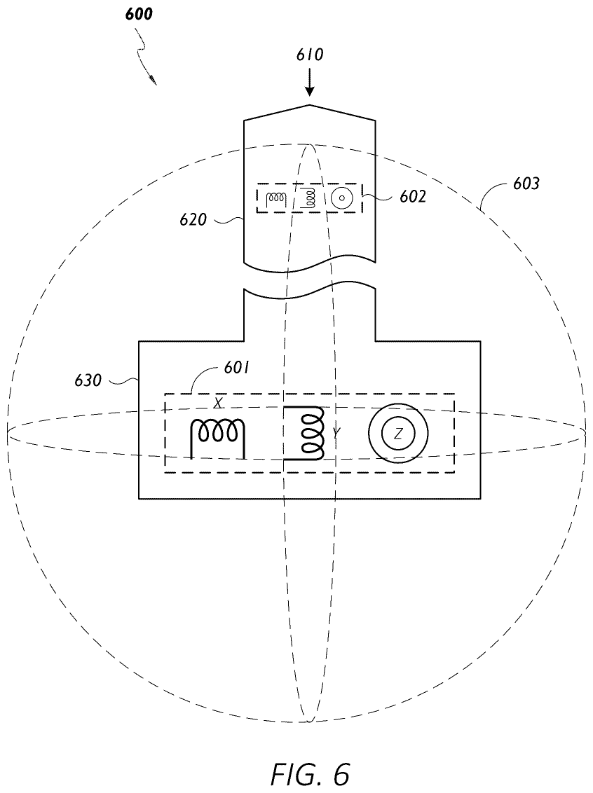

FIG. 6 shows another example medical instrument 600, according to some implementations. In the example of FIG. 6, the medical instrument 600 is depicted as a percutaneous access needle. In some implementations, the medical instrument 600 may be one example of the medical instrument 500 of FIG. 5.

The medical instrument includes 600 a trocar 610, a cannula 620, and a hub 630 which forms the base of the needle. For simplicity, the cannula 620 is shown in truncated form. The hub 630 includes an EM sensor 601 that can be used for determining a position of the medical instrument 600 in the presence of magnetic fields (such as described with reference to FIGS. 1-4). In some implementations, the EM sensor 601 may be one example of the EM sensor 501 of FIG. 5. More specifically, the EM sensor 601 may produce sensor data indicating a position of the hub 630 with 6 degrees of freedom (such as by a position vector and quaternion). In the example of FIG. 6, the EM sensor 601 is shown to include a trio of inductive coils each positioned parallel to a different plane or axis of rotation (labeled “X,” “Y,” and “Z” in cartesian coordinate space). Although depicted as separate and discrete inductors in FIG. 6, the coils may be combined or otherwise wrapped around a single core in some other implementations (such as a 3-axis magnetometer).

The medical instrument 600 further includes another sensor 602 disposed in the trocar 610. In some implementations, the sensor 602 may be one example of any of the sensors 502A-502D of FIG. 5. Although not shown for simplicity, the medical instrument 600 may include additional sensors (similar or identical to the sensor 602) disposed along the length of the cannula 620. In some implementations, the sensor 602 may be disposed on an inner wall or surface of the medical instrument 600. In some other implementations, the sensor 602 may be disposed on an outer surface of the medical instrument 600. In the example of FIG. 6, the sensor 602 includes a trio of inductive coils each positioned parallel to a different plane or axis of rotation (similar to the EM sensor 601 in the hub 630). Although depicted as separate and discrete inductors in FIG. 6, the coils may be combined or otherwise wrapped around a single core in some other implementations (such as a 3-axis magnetometer).

In some aspects, the sensor 601 may sense the position and orientation of the hub 630 based on magnetic fields generated by an EM field generator (such as the EM field generator 180 of FIGS. 1-4) and the sensor 602 may sense the position and orientation of the trocar 610 based on the same magnetic fields generated by the EM field generator. More specifically, the sensors 601 and 602 may concurrently convert the magnetic fields to a time-varying current or signal which can be used to determine positions of the trocar 610 and the hub 630, respectively, on a common or global coordinate frame. In such aspects, the shape of the cannula 620 can be determined based on global coordinates of the trocar 610 and the hub 630. For example, the positions and/or orientations of the trocar 610 and the hub 630 may indicate a bend or curvature of the cannula 620. Alternatively, the bend or curvature of the cannula 620 can be determined or estimated by measuring a distance between the position of the trocar 610 and the position of the hub 630 and comparing the measured distance to a known length of the medical instrument 600.

In some other aspects, the sensors 601 and 602 may be configured to provide a localized EM tracking system. For example, the individual coils in the sensor 601 may be driven or pulsed with a current to induce a set of (time- or position-varying) magnetic fields 603 that can be detected by the sensor 602 in the trocar 610. The individual coils may receive the current pulses sequentially or simultaneously (such as with different frequencies or waveform shapes than the EM fields produced by the EM field generator). The current induced in the sensor 602 indicates a local position and orientation of the trocar 610 relative to a position and orientation of the hub 630 (based on prior calibration of the generator field) which can be used to determine the shape of the cannula 620.

In some aspects, the sensor 601 in the hub 630 may operate in a “passive mode,” in which the sensor 601 senses the magnetic fields generated by the EM field generator to determine the position and orientation of the hub 630, or a “generator mode” in which the sensor 601 generates magnetic fields that can be used to determine the position and orientation of the trocar 610. In some implementations, the sensor 601 may be configured to alternate between the passive mode and the generator mode in a time- or frequency-multiplexed fashion. Alternatively, the hub 630 may include another sensor (similar if not identical to the sensor 601) configured to operate in the passive mode while the sensor 601 operates in the generator mode.

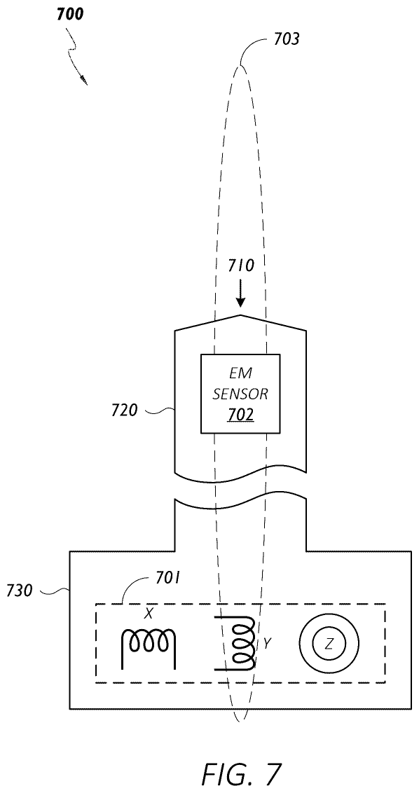

FIG. 7 shows another example medical instrument 700, according to some implementations. In the example of FIG. 7, the medical instrument 700 is depicted as a percutaneous access needle. In some implementations, the medical instrument 700 may be one example of the medical instrument 500 of FIG. 5.

The medical instrument includes 700 a trocar 710, a cannula 720, and a hub 730 which forms the base of the needle. For simplicity, the cannula 720 is shown in truncated form. The hub 730 includes an EM sensor 701 that can be used for determining a position of the medical instrument 700 in the presence of magnetic fields (such as described with reference to FIGS. 1-4). In some implementations, the EM sensor 701 may be one example of the EM sensor 501 of FIG. 5. More specifically, the EM sensor 701 may produce sensor data indicating a position of the hub 730 with 6 degrees of freedom (such as by a position vector and quaternion). In the example of FIG. 7, the EM sensor 701 is shown to include a trio of inductive coils each positioned parallel to a different plane or axis of rotation (labeled “X,” “Y,” and “Z” in cartesian coordinate space). Although depicted as separate and discrete inductors in FIG. 7, the coils may be combined or otherwise wrapped around a single core in some other implementations (such as a 3-axis magnetometer).

The medical instrument 700 further includes another sensor 702 disposed in the trocar 710. In some implementations, the sensor 702 may be one example of any of the sensors 502A-502D of FIG. 5. Although not shown for simplicity, the medical instrument 700 may include additional sensors (similar or identical to the sensor 702) disposed along the length of the cannula 720. In some implementations, the sensor 702 may be disposed on an inner wall or surface of the medical instrument 700. In some other implementations, the sensor 702 may be disposed on an outer surface of the medical instrument 700. In the example of FIG. 7, the sensor 702 may be any EM sensor having 6 degrees of freedom (similar to the EM sensor 501 of FIG. 5).

In some aspects, the sensor 701 may sense the position and orientation of the hub 730 based on magnetic fields generated by an EM field generator (such as the EM field generator 180 of FIGS. 1-4) and the sensor 702 may sense the position and orientation of the trocar 710 based on the same magnetic fields generated by the EM field generator. More specifically, the sensors 701 and 702 may concurrently convert the magnetic fields to a time-varying current or signal which can be used to determine positions of the trocar 710 and the hub 730 on a common or global coordinate frame. In such aspects, the shape of the cannula 720 can be determined based on global coordinates of the trocar 710 and the hub 730 (such as described with reference to FIG. 6).

In some other aspects, the sensors 701 and 702 may be configured to provide a localized EM tracking system. More specifically, the sensor 702 may be driven or pulsed with a current to induce a magnetic field 703 that can be detected by the sensor 701 in the hub 730. The current induced in the sensor 701 indicates a local position and orientation of the trocar 710 relative to a position and orientation of the hub 730 (based on prior calibration of the generator field) which can be used to determine the shape of the cannula 720. In some implementations, the sensor 701 may be configured to alternately sense the magnetic fields generated by the EM field generator (such as to determine the position and orientation of the hub 730 in the global coordinate frame) and the magnetic fields generated by the sensor 702 (such as to determine the relative position and orientation of the trocar 710), for example, by driving the current onto the sensor 702 in a time- or frequency-multiplexed manner.

Aspects of the present disclosure further recognize that, given the small coil sizes and low signal levels needed to implement the sensors in the medical instruments 500-700 of FIGS. 5-7, respectively, the induced voltage in these sensors can be sampled with an analog-to-digital converter (ADC) inside the hub of the medical instrument. Thus, in some aspects, an ADC may be integrated into the hub of a medical instrument (such as any of the medical instruments 500-700 of FIGS. 5-7, respectively) to convert the analog signals received from any of the sensors to the digital domain. Accordingly, the medical instrument can transmit a digital (rather than analog) signal back to the control system (such as the control system 140 of FIG. 1). This reduces noise in the deflection measurement, and potentially allows for signal transport with the needle hub sensor data on a common digital bus.

Among other advantages, aspects of the present disclosure enable the shape of a percutaneous access instrument to be estimated at any time during a medical procedure. Displaying the shape of the instrument may enable shape-informed percutaneous access and provide more insight as to whether critical anatomy has been transited as well as the spatial relationship between the instrument insertion site and the instrument tip. Moreover, larger and more accurate sensors (which are generally more robust against metal distortion) can be placed in the hub of the instrument, while shaft deflection allows the medical system to continue tracking the tip position. In the most basic, low-cost configuration, the instrument shape can be estimated with a single sensor placed at an axial location in the instrument shaft. By using strain gauges to sense the shape of the instrument, the localization of the instrument tip can be free of electromagnetic interference or distortion from metallic objects in the clinical environment.

As described with reference to FIGS. 1-4, the medical instruments 500-700 of FIGS. 5-7, respectively, may be manually driven by a physician or with the assistance of a robotic system. Where the needle is inserted manually, aspects of the present disclosure may still leverage instrument shape information in certain parts of the procedure. For example, instrument shape presents a more accurate estimate of percutaneous access position and angle, which is not available as real-time information in many existing medical systems. In some implementations, a controller (such as the control system 140 of FIG. 1) may display a graphical interface (such as the percutaneous access interface 144) that provides more accurate guidance for inserting the instrument into an anatomy based on the instrument shape information (such as to compensate for changes in the trajectory of the instrument based on the shape or bend of the instrument).

In some aspects, the controller may use the needle position and/or shape in determining how to position other medical instruments. For example, in some implementations, the instrument shape estimates may be used to automate a percutaneous antegrade ureteroscopy (PAU) arm alignment algorithm. For example, the PAU arm may automatically move to a particular pose that is close, but with some safety buffer, to the percutaneous access point.

In some other aspects, the needle may be robotically controlled or manipulated. For example, the needle may be coupled to a robotic arm (such as any of the robotic arms 112 of FIG. 1) that can insert the needle into the anatomy. In some implementations, the controller may use the instrument shape information as feedback for controlling the robotic arm(s), for example, to ensure that the instrument does not stray from the intended trajectory while being inserted into the anatomy.

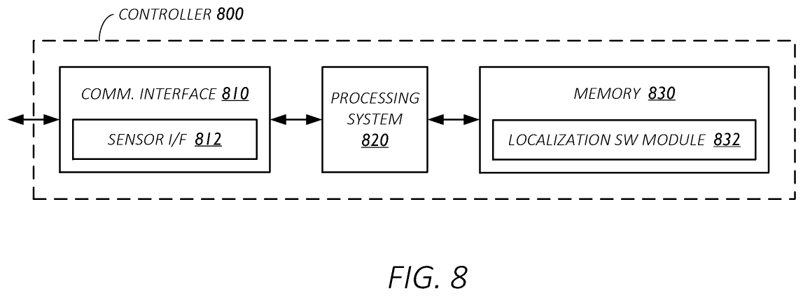

FIG. 8 shows a block diagram of an example controller 800 for a medical system, according to some implementations. In some implementations, the controller 800 may be one example of the control system 140 of FIGS. 1-4. More specifically, the controller 800 is configured for localizing medical instruments, for example, by tracking a pose and/or shape of the instruments.

The controller 800 includes a communication interface 810, a processing system 820, and a memory 830. The communication interface 810 is configured to communicate with one or more components of the medical system. More specifically, the communication interface 810 includes a sensor interface (I/F) 812 for communicating with one or more sensors of the medical system (such as any of the sensors 501 or 502A-502D of FIG. 5, any of the sensors 601 or 602 of FIG. 6, and/or any of the sensors 701 or 702 of FIG. 7). In some implementations, the sensor I/F 812 may be configured to receive sensor data from a sensor disposed on an instrument having a distal portion configured to be inserted into an anatomy, where the sensor data indicates a position or orientation of a proximal portion of the instrument.

The memory 830 may include a non-transitory computer-readable medium (including one or more nonvolatile memory elements, such as EPROM, EEPROM, Flash memory, or a hard drive, among other examples) that may store a localization software (SW) module 832 to determine a position or orientation of the distal portion of the instrument based at least in part on the received sensor data and a known length of the instrument. The localization SW module 832 includes instructions that, when executed by the processing system 820, causes the controller 800 to perform the corresponding functions.

The processing system 820 may include any suitable one or more processors capable of executing scripts or instructions of one or more software programs stored in the controller 800 (such as in the memory 830). For example, the processing system 820 may execute the localization SW module 832 to determine a position or orientation of the distal portion of the instrument based at least in part on the received sensor data and a known length of the instrument.

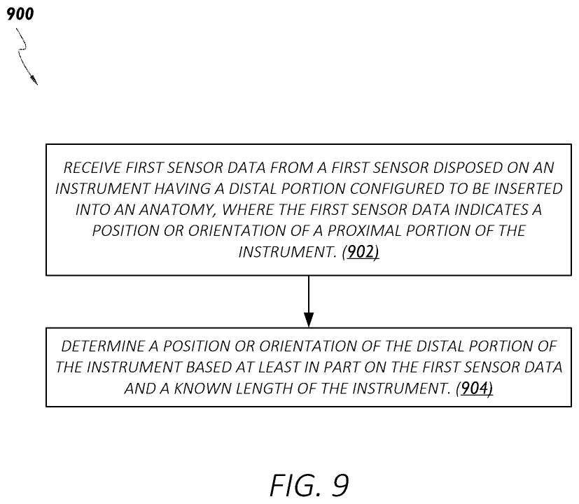

FIG. 9 shows an illustrative flowchart depicting an example operation 900 for localizing medical instruments, according to some implementations. In some implementations, the example operation 900 may be performed by a controller for a medical system such as the controller 800 of FIG. 8 or the control system 140 of FIG. 1.

The controller receives first sensor data from a first sensor disposed on an instrument having a distal portion configured to be inserted into an anatomy, where the first sensor data indicates a position or orientation of a proximal portion of the instrument (902). The controller further determines a position or orientation of the distal portion of the instrument based at least in part on the first sensor data and a known length of the instrument (904).

In some aspects, the controller may further determine a shape or bend of a shaft of the instrument between the distal portion and the proximal portion, where the position or orientation of the distal portion of the instrument is further determined based on the shape or bend of the shaft. In some implementations, the instrument may include one or more second sensors disposed along the shaft and configured to produce second sensor data indicating the shape or bend of the shaft.

In some other implementations, the controller may drive the first sensor with current that induces one or more magnetic fields; detect current induced by the one or more magnetic fields in a second sensor disposed on the distal portion of the instrument; and determine a position or orientation of the distal portion of the instrument relative to the position or orientation of the proximal portion based at least in part on the detected current from the second sensor, where the shape or bend of the shaft is determined based on the relative position or orientation of the distal portion of the instrument.

In some other implementations, the controller may drive a second sensor disposed on the proximal portion of the instrument with current that induces one or more magnetic fields; detect current induced by the one or more magnetic fields in a third sensor disposed on the distal portion of the instrument; and determine a position or orientation of the distal portion of the instrument relative to the position or orientation of the proximal portion based at least in part on the detected current from the third sensor, where the shape or bend of the shaft is determined based on the relative position or orientation of the distal portion of the instrument.

Still further, in some implementations, the controller may drive a second sensor disposed on the distal portion of the instrument with current that induces a magnetic field; detect current induced in the first sensor by the magnetic field; and determine a position or orientation of the distal portion of the instrument relative to the position or orientation of the proximal portion based at least in part on the detected current from the first sensor, where the shape or bend of the shaft is determined based on the relative position or orientation of the distal portion of the instrument.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A system comprising:
an instrument having a distal portion configured to be inserted into an anatomy;
a first sensor disposed on a proximal portion of the instrument; and
a controller configured to:
receive first sensor data from the first sensor indicating a position or orientation of the proximal portion of the instrument; and
determine a position or orientation of the distal portion of the instrument based at least in part on the first sensor data and a known length of the instrument.

2. The system of claim 1, wherein the first sensor comprises an electromagnetic (EM) sensor having at least six degrees of freedom.

3. The system of claim 1, wherein the controller is further configured to:
determine a shape or bend of a shaft of the instrument between the distal portion and the proximal portion, the position or orientation of the distal portion of the instrument further being determined based on the shape or bend of the shaft.

4. The system of claim 3, wherein the controller is further configured to:
display a graphical interface that provides guidance for inserting the instrument into the anatomy based at least in part on the shape or bend of the shaft.

5. The system of claim 3, wherein the system further comprises a robotic arm coupled to the instrument and configured to insert the instrument into the anatomy, the controller being further configured to:
control the insertion of the instrument by the robotic arm based at least in part on the shape or bend of the shaft.

6. The system of claim 3, wherein the system further comprises a robotic arm coupled to another medical instrument, the controller being further configured to:
control a pose of the robotic arm based at least in part on the shape or bend of the shaft.

7. The system of claim 3, further comprising:
one or more second sensors disposed along the shaft and configured to produce second sensor data indicating the shape or bend of the shaft.

8. The system of claim 3, further comprising:
a second sensor disposed on the distal portion of the instrument.

9. The system of claim 8, wherein the second sensor comprises an EM sensor having at least six degrees of freedom, the controller being further configured to:
drive the first sensor with current that induces one or more magnetic fields;
detect current induced in the second sensor by the one or more magnetic fields; and
determine a position or orientation of the distal portion of the instrument relative to the position or orientation of the proximal portion based at least in part on the detected current from the second sensor, the shape or bend of the shaft being determined based on the relative position or orientation of the distal portion of the instrument.

10. The system of claim 9, wherein the controller is configured to alternately receive the first sensor data from the first sensor and drive the first sensor with the current that induces the plurality of magnetic fields.

11. The system of claim 8, wherein the system further comprises a third sensor disposed on the proximal portion of the instrument, the controller being further configured to:
drive the third sensor with current that induces one or more magnetic fields;
detect current induced in the second sensor by the one or more magnetic fields; and
determine a position or orientation of the distal portion of the instrument relative to the position or orientation of the proximal portion based at least in part on the detected current from the second sensor, the shape or bend of the shaft being determined based on the relative position or orientation of the distal portion of the instrument.

12. The system of claim 8, wherein the controller is further configured to:
drive the second sensor with current that induces a magnetic field;
detect current induced in the first sensor by the magnetic field; and
determine a position or orientation of the distal portion of the instrument relative to the position or orientation of the proximal portion based at least in part on the detected current from the first sensor, the shape or bend of the shaft being determined based on the relative position or orientation of the distal portion of the instrument.

13. The system of claim 12, wherein the controller is configured to alternately receive the first sensor data from the first sensor and drive the second sensor with the current that induces the magnetic field.

14. The system of claim 1, further comprising:
an analog-to-digital converter (ADC) disposed on the proximal portion of the instrument and configured to convert the first sensor data from an analog domain to a digital domain so that the controller receives the first sensor data in the digital domain.

15. A method for localizing medical instruments, comprising:
receiving first sensor data from a first sensor disposed on an instrument having a distal portion configured to be inserted into an anatomy, the first sensor data indicating a position or orientation of a proximal portion of the instrument; and
determining a position or orientation of the distal portion of the instrument based at least in part on the first sensor data and a known length of the instrument.

16. The method of claim 15, further comprising:
determining a shape or bend of a shaft of the instrument between the distal portion and the proximal portion, the position or orientation of the distal portion of the instrument further being determined based on the shape or bend of the shaft.

17. The method of claim 16, wherein the instrument comprises one or more second sensors disposed along the shaft and configured to produce second sensor data indicating the shape or bend of the shaft.

18. The method of claim 16, further comprising:
driving the first sensor with current that induces one or more magnetic fields;
detecting current induced by the one or more magnetic fields in a second sensor disposed on the distal portion of the instrument; and
determining a position or orientation of the distal portion of the instrument relative to the position or orientation of the proximal portion based at least in part on the detected current from the second sensor, the shape or bend of the shaft being determined based on the relative position or orientation of the distal portion of the instrument.

19. The method of claim 16, further comprising:
driving a second sensor disposed on the proximal portion of the instrument with current that induces one or more magnetic fields;
detecting current induced by the one or more magnetic fields in a third sensor disposed on the distal portion of the instrument; and
determining a position or orientation of the distal portion of the instrument relative to the position or orientation of the proximal portion based at least in part on the detected current from the third sensor, the shape or bend of the shaft being determined based on the relative position or orientation of the distal portion of the instrument.

20. The method of claim 16, further comprising:
driving a second sensor disposed on the distal portion of the instrument with current that induces a magnetic field;
detecting current induced in the first sensor by the magnetic field; and
determining a position or orientation of the distal portion of the instrument relative to the position or orientation of the proximal portion based at least in part on the detected current from the first sensor, the shape or bend of the shaft being determined based on the relative position or orientation of the distal portion of the instrument.
